(19) United States
(12) Patent Application Publication
(10) Pub. No.: US 2009/0171170 A1
(43) Pub. Date: Jul. 2, 2009

(54) Medical Monitoring With Portable Electronic Device System And Method

(75) Inventors: Li Li, Milpitas, CA (US); Scott Amundson, Oakland, CA (US); Corydon A. Hinton, Oakland, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/343,792

(22) Filed: Dec. 24, 2008

Related U.S. Application Data
(60) Provisional application No. 61/009,452, filed on Dec. 28, 2007.

(51) Int. Cl. A61B 5/00

(52) U.S. Cl. 600/301

(57) ABSTRACT

In an embodiment, a system is provided that includes a sensor configured to monitor a physiological parameter of the patient. The sensor is further configured to communicate with a portable electronic device configured to communicate over a wireless network. A method of operation of a sensor is provided that includes receiving data from a sensor configured to sense a physiological parameter and determining the physiological parameter from the data. A method of operation of a portable electronic device is also provided.

### RELATED APPLICATIONS

This application claims priority to U.S. Provisional Application No. 61/009,452, filed Dec. 28, 2007, and is incorporated herein by reference in its entirety.

### BACKGROUND

The present disclosure relates generally to medical devices and, more particularly, to medical sensors and monitoring devices.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient.

Pulse oximeters typically utilize a non-invasive sensor that is placed on or against a patient's tissue that is well perfused with blood, such as a patient's finger, toe, forehead or earlobe. The pulse oximeter sensor emits light and photoelectrically senses the absorption and/or scattering of the light after passage through the perfused tissue. The data collected by the sensor may then be used to calculate one or more of the above physiological characteristics based upon the absorption or scattering of the light. More specifically, the emitted light is typically selected to be of one or more wavelengths that are absorbed or scattered in an amount related to the presence of oxygenated versus de-oxygenated hemoglobin in the blood. The amount of light absorbed and/or scattered may then be used to estimate the amount of the oxygen in the tissue using various algorithms.

Pulse oximeters and other medical devices are typically mounted on stands that are positioned around a patient's bed or around an operating room table. When a caregiver desires to command the medical device (e.g., program, configure, and so-forth) the caregiver must manipulate controls or push buttons on the monitoring device itself. The monitoring device typically provides results or responses to commands on a Liquid Crystal Diode (‘LCD’) screen mounted in an externally visible position within the medical device. Patient data, alerts, and other information may be displayed on the monitor directly, or may be transmitted over a wired link to a central computer monitored by caregivers.

This conventional configuration, however, has several disadvantages. For example, some patients may need monitoring outside of a hospital environment, and such monitors are typically too expensive and complex for home use. Also, for ambulatory patients, conventional monitors are too heavy and bulky to be worn or constantly moved around to follow a patient. Additionally, if the monitoring occurs outside of the hospital environment, a caregiver may not receive an immediate update or alert on the patient's condition. Further, the monitor and/or sensor may require frequent calibration or software upgrades that may be difficult to provide outside of a hospital or medical environment.

In some instances, a patient may be bedridden at home or in another location outside of a hospital or medical environment. In those instances, although portability of the sensor and monitor may not be as large of a concern, a caregiver will still require the ability to monitor the patient's condition and be aware of any alerts related to the physiological parameters of the patient. In such cases, 24 hour monitoring by the patient or other personnel at the patient's location may not be possible.

### SUMMARY

Certain aspects generally commensurate with the originally claimed invention are set forth below. It should be understood that these aspects are presented merely to provide the reader with a brief summary of certain forms that any claimed invention might take and that these aspects are not intended to limit the scope of any claimed invention. Indeed, any invention claimed presently or in the future may encompass a variety of aspects that may not be set forth below.

In one embodiment, a system is provided that includes a sensor configured to monitor a physiological parameter, wherein the sensor is further configured to communicate with a portable electronic device.

A method of operating a sensor is provided that includes sensing a physiological parameter of a patient and providing a signal correlative to the physiological parameter to a portable electronic device.

A method of operating a portable electronic device is provided that includes receiving data from a sensor configured to sense a physiological parameter and determining the physiological parameter from the data.

In another embodiment, a system is provided that includes a monitor configured to connect to a sensor configured to monitor a physiological parameter, wherein the monitor is further configured to communicate with a portable electronic device.

### BRIEF DESCRIPTION OF THE DRAWINGS

Advantages of the disclosure may become apparent upon reading the following detailed description and upon reference to the drawings in which:

FIG. 1 is a diagrammatical representation of a portable medical sensor and device in accordance with an embodiment of the present disclosure;

FIG. 2 is a diagrammatical representation of the portable medical device of FIG. 1 in a network system in accordance with an embodiment of the present disclosure;

FIG. 3 is a flowchart illustrating an exemplary technique for operating the portable medical device in accordance with an embodiment of the present disclosure;

FIG. 4 is a block diagram of the portable medical device in accordance with an embodiment of the present disclosure.

### DETAILED DESCRIPTION OF SPECIFIC EMBODIMENTS

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

It is desirable to provide a patient sensor and a monitor, such as for use in pulse oximetry, which is integrated with a portable electronic device having a remote communication interface, e.g. a mobile phone or wireless Internet-enabled handheld device, to form a portable medical device capable of remote communications. In accordance with some aspects of the present technique, the sensor may communicate with a portable medical device either through a cable or a wireless interface, and the portable medical device may analyze information received from the sensor. Further, the portable medical device may send this information, alerts or any other data to a remote computer or a caregiver via the remote communication interface. Additionally, the portable medical device may receive calibration information, software upgrades, or any other data from a remote location or caregiver and use such information or provide it to the sensor.

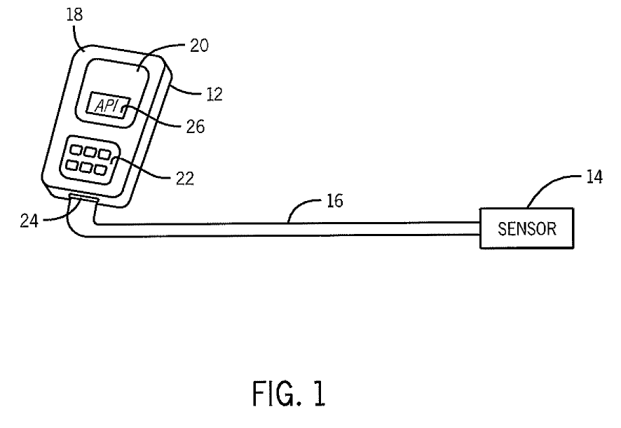

Referring now to FIG. 1, a portable medical device 12 is shown connected to a sensor 14, according to an embodiment. The portable medical device 12 may be a cellular phone, a personal data assistant, a GPS device, any wireless handheld device, or any other portable medical device 12, or any other device capable of wireless communication. Additionally, the portable medical device 12 may be a combination of any of the devices listed above.

In an embodiment, the portable medical device 12 may be connected to the sensor 14 via a sensor cable 16, to facilitate communication between the portable medical device 12 and the sensor 14. In other embodiments, the sensor 14 may communicate with the portable medical device 12 wirelessly, such as through the use of wireless technology such as radio, infrared, or optical signals In an embodiment, the sensor 14 may communicate with the portable medical device 12 via a wireless communication protocol such as Bluetooth. As will be appreciated, the portable medical device 12, sensor 14 and/or the sensor cable 16 may include or incorporate one or more integrated circuit devices or electrical devices, such as a memory, processor, etc., that may facilitate or enhance communication between the sensor 14 and the portable medical device 12. Likewise the sensor cable 16 may be an adaptor cable, with or without an integrated circuit or electrical device, for facilitating communication between the sensor 10 and the portable medical device 12.

In an embodiment, the portable medical device 12 may include an enclosure 18, a display 20, and one or more user inputs 22. The user inputs 22 may include a keyboard, and/or any number of knobs, buttons, dials, or any other suitable input device. The display 20 may display a user interface and various indicators for the portable medical device 12, the sensor 14, and/or the sensor cable 16. In some embodiments, the display 20 may comprise a touchscreen, and user inputs may be available through the touchscreen, with or without the inclusion of user inputs 22. The portable medical device 12 may include a connector 24 configured to connect to the sensor cable 16. The connector 24 may be a Universal serial bus (USB) connector, FireWire (IEEE 1394) connector, or any other suitable connector. Alternatively, the connector 24 may be custom designed to interface with the sensor cable 16 such that other cables or devices may not be used with the connector 24.

The portable medical device 12 may run any suitable operating system, such as Symbian OS, Linux, Windows CE, Palm OS, etc., or the portable medical device 12 may run a custom operating system suitably designed for the application described herein. Additionally, to support software that may be used with the sensor 14, such as the user interface and sensor interface, the portable medical device 12 may include an application programming interface (API) 26. The API 26 may facilitate development of the user interface, sensor interface, and other software by providing a set of requests, calls, methods, or other useful programming interfaces that allow easier usage of the resources provided by the portable medical device 12. For example, the API 26 may provide details for implementing the user interface, such as how to display a window, move a window, or display user interface objects such as buttons, icons, etc. In addition, with regard to the sensor interface, the API 26 may provide details on how the portable medical device 12 may receive, send, and process information to/from the sensor 14 and/or the sensor cable 16. In one embodiment, the APT 26 may comprise the Binary Runtime Environment for Wireless (BREW). In another embodiment, the API 26 may comprise Java Platform Micro Edition (J2ME). However, in some embodiments an API 26 may not be provided.

The sensor 14 may be any sensor configured to monitor a physiological parameter and may be connected to a body part (e.g., finger, forehead, toe, or earlobe) of a patient or a user. The sensor 14 may be configured to be clipped onto a finger or earlobe or may be configured to be secured with tape or another static mounting technique. For example, as a pulse oximetry sensor, the sensor 14 may clip onto a patient or user's finger and may be configured to emit signals or waves into the patient's or user's tissue and detect these signals or waves after dispersion and/or reflection by the tissue. For example, the sensor 14 may be configured to emit light from two or more light emitting diodes (“LEDs”) into pulsatile tissue (e.g., finger, forehead, toe, or earlobe) and then detect the transmitted light with a light detector (e.g., a photodiode or photo-detector) after the light has passed through the pulsatile tissue. In other embodiments, the sensor may be a reflectance-type pulse oximetry sensor, an electrocardiogram (EKG), a blood sugar (glucose) sensor, a blood pressure sensor, a temperature sensor, or any other sensor configured to monitor a physiological parameter. Indeed) the sensor 14 could include an implanted device, such as a pacemaker or defibrillator that communicates with the portable device wirelessly. The sensor 14 may read data from a patient or user and send the data to the portable medical device 12. Additionally, the sensor 14 may receive data from the portable medical device 12, such as calibration information or updated firmware. The sensor 14 may also receive power from the portable medical device 12 via sensor cable 16, or the sensor 14 may include a battery to provide power.

As described further below, implementation of the sensor 14 with the portable medical device 12 may provide for a variety of monitoring and alert functions. For example, in one embodiment the portable medical device 12 may compile a summary report of data received by the sensor, and the portable medical device 12 may send reports to a remote location at a specified time interval, such as every 10 minutes, every hour, every day, etc. Alteratively, the portable medical device 12 may continuously send data to the remote location. Further, the sensor 14 may be configured to continuously monitor and/or periodically monitor the physiological characteristic of a user. For example, if the sensor 14 is a pulse oximetry sensor, it would likely provide substantially continuous monitoring of the patient. On the other hand, if the sensor 14 is a blood pressure sensor, it would likely provide only periodic measurements.

In addition to monitoring, the portable medical device 12 may use information received from the sensor 14 to provide alarms, alerts, or any other notification based on the status of the physiological parameter of the user monitored by the sensor 12. As discussed further below, in an embodiment, if the physiological parameter is above or below a specified threshold, the portable medical device 12 may provide an alarm or any other audio or visual notification, thus notifying the user of the abnormal condition. Additionally, or alternatively, the portable medical device 12 may send an alert to remote location, such as to a caregiver responsible for monitoring or treating the patient or user. For example, if the portable medical device 12 includes a cellular telephone, a call and/or text message could be sent to a caregiver to provide information related to the patient's condition. Similarly, if the portable medical device 12 includes wireless Internet capability, an email could be sent to a caregiver to provide information related to the patient's condition. Thus, the status of a user's physiological parameter and any abnormal conditions related to those physiological parameters may be monitored, and a caregiver may be automatically notified without any intervention from the patient or user using the portable medical device 12 and the sensor 14.

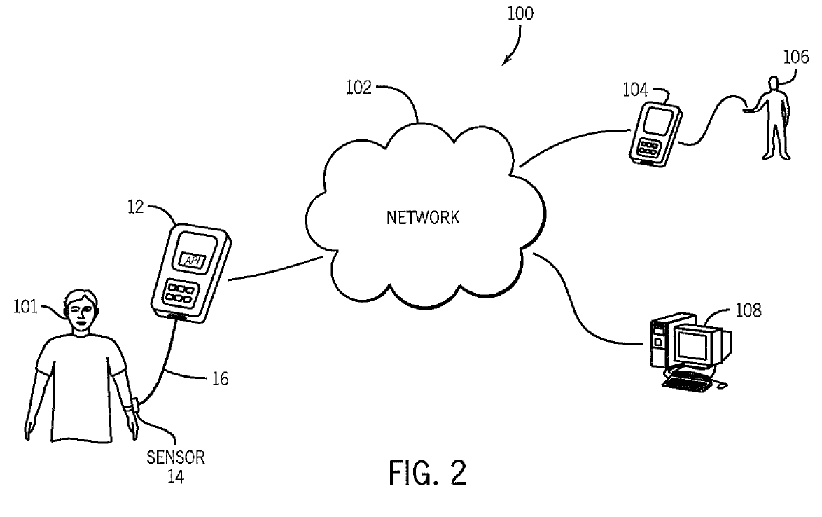

To describe this capability more clearly, FIG. 2 depicts in an embodiment a system 100 that includes the portable medical device 12 and sensor 14 in communication with other devices over a network 102. The sensor 14 may be connected to a user 101, such as clipped to a finger. In one embodiment, the portable medical device 12 may be connected to the network 102 wirelessly, and the network 102 may be any wireless network. For example, in such an embodiment the network 102 may be Ethernet, Wi-Fi (IEEE 802.11 standards), WiMax, GSM, 3GSM, GPRS, PCS, TDMA, CDMA, EV-DO, or any other suitable network. In other embodiments, the portable medical device 12 may be connected to the network via a network cable. Additionally, the network 102 may be any local area network (LAN), wide-area network (WAN), or the Internet.

Other devices may be connected to the network 102 and may send or receive data from the portable medical device 12. The network may include other portable electronic devices 104, such as those used or carried by a caregiver 106. Additionally, the network may include a remote computer 108, such as a desktop, server, database server, or any other computer at a remote location.

As discussed herein, in response to data received from the sensor 14, the portable medical device 12 may contact other devices on the network. For example, if the user 101 experiences an abnormal condition, such a change in a physiological parameter of the user may be detected by the sensor 14 and received by the portable medical device 12. Based on this data, if the portable medical device 12 determines that an alert should be sent to the caregiver 106, the portable medical device 12 may send an alert message over the network 102 to the caregiver's portable electronic device 104. A caregiver 106 carrying the portable electronic device 104 or sitting at the computer 108 may then receive the alert message and take the appropriate action, such as contacting the user 101 or sending emergency personnel.

Additionally, if one of the functions performed by the portable medical device 12 is generation of summary reports of data received from the sensor 14, the portable medical device 12 may send those reports to the remote computer 108 for storage and archiving. A caregiver 106 may then review a user's history on the remote computer 108, and then take the appropriate action. For example, a user's history reviewed on the remote computer 108 may indicate that a change in treatment is necessary, such as a change in medication, scheduling of a checkup, an increase or reduction in therapies, et cetera.

In some instances, the portable medical device 12 may receive data from another portable electronic device 104 or from the remote computer 108. For example, the portable medical device 12 may receive software updates from the remote computer 108. The software updates may include updates to the user interface or the sensor interface on the portable medical device 12. In addition to software updates, the portable medical device 12 may receive calibration information from the remote computer 108. For example, if the sensor 14 or portable medical device 12 indicates that recalibration is necessary, it may receive the appropriate algorithms and calibration coefficients from the remote computer 108. It should be appreciated that in some embodiments the portable electronic device may be directly connected to a local computer via a wireless or physical connection and may receive the updates, calibration information, or any other information through this means of connection.

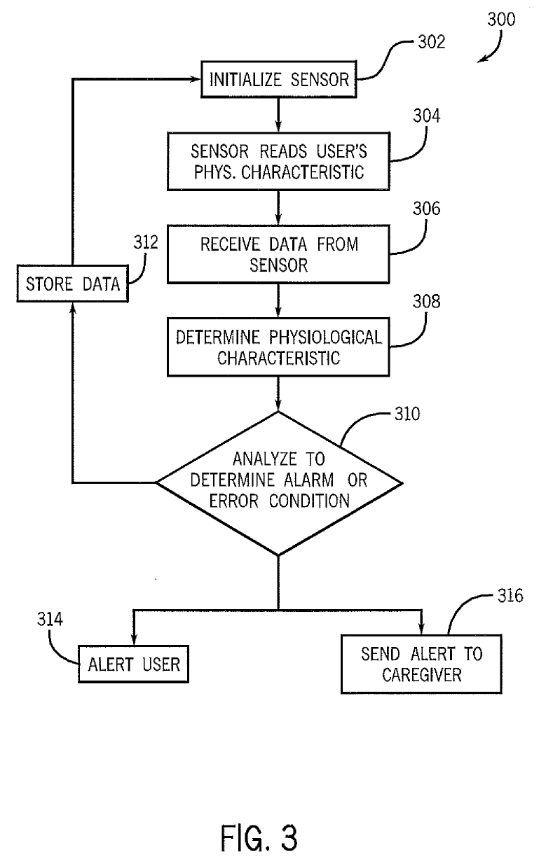

Turning now to the operation of the devices discussed above, FIG. 3 depicts a flowchart 300 illustrating the operation of a sensor and portable electronic device in accordance with an embodiment. The sensor may be connected to the portable electronic device and initialized (302). Initialization of the sensor may include detection of the type of sensor, e.g. pulse oximeter, EKG, temperature, etc., by the portable electronic device and selection of the appropriate calibration algorithms and calibration coefficients. Additionally, a sensor may be initialized depending on the specifics of the patient using the sensor. For example, information about the patient, such as height, weight, skin color, race, age, or any other characteristic may be stored in the portable electronic device for use when the sensor is initialized.

Once the sensor is initialized and ready, the sensor may read the patient's physiological parameters (block 304). For example, in one embodiment in which the sensor is a pulse oximeter, the reading process may include passing light through a patients tissue such as a finger, and detecting the light after it is been absorbed and/or scattered by various tissue constituents as described above. Once the sensor has acquired data from the patient, this data may be received by the portable electronic device (block 306). From this data, and using the calibration algorithms and calibration coefficients that may have been selected upon initialization of the sensor, the portable electronic device may determine a physiological characteristic of the patient (block 308). For example, in the case of pulse oximetry, the portable electronic device may determine the patient's blood-oxygen saturation levels

To determine the status of the patient monitored by the sensor, the physiological characteristic determined by the portable electronic device may be analyzed to determine if an alarm or error condition exists (decision block 310). For example, the physiological characteristic may be compared to a threshold value. In some embodiments, the threshold value may represent a minimum value, a maximum value, or a baseline value. Thus, the comparison may determine if the physiological characteristic is below the threshold value, above the threshold value, or beyond a specified deviation from the baseline value. If the comparison determines that the physiological characteristic is normal, then the value of the physiological characteristic may be stored (block 312) and the sensor may continue monitoring.

If the comparison to a threshold value determines that the value of the physiological characteristic is abnormal, then the portable electronic device may take the appropriate actions. For example, the portable electronic device may alert the patient of the device (block 314), such as by providing an audio and/or visual notification. The patient may then take appropriate actions, such as calling medical personnel, taking medication, etc. In addition, or alternatively, the portable electronic device may send an alert to a caregiver (block 316) via communication with another portable electronic device over a network, as described above. In response, the caregiver may call or send a message to the user's portable electronic device suggesting instructions or actions to take in response to the abnormal physiological condition.

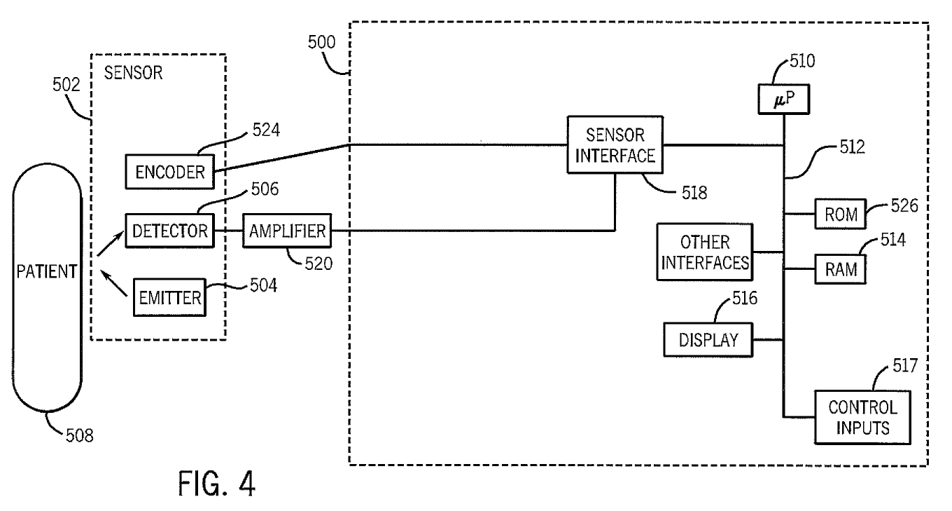

FIG. 4 is a block diagram of one embodiment of a portable electronic device 500 and a sensor 502 that form a portable medical device 503. As described below, the sensor 502 may include an emitter 504 and a detector 506, such as for use with pulse oximetry techniques. However, any sensor capable of reading a physiological parameter may be used with the patient monitor 500 and with the embodiments described.

Turning now to operation of the sensor 502 and the portable electronic device 500 in which the sensor 502 is a pulse oximetry sensor, light from emitter 504 passes into the tissue of a patient 508, and is scattered and detected by detector 506. The sensor 502 is connected to the portable electronic device 500. As discussed above, the sensor 502 may be physically connected to the portable electronic device 500 or may communicate via wireless technology and communication protocols. The portable electronic device 500 may include a microprocessor 510 connected to an internal bus 512, and may include a RAM memory 514 and a display 516 connected to the bus 512. The portable electronic device 500 may also include user inputs 517. As discussed above, the user inputs 517 may include a keyboard, and/or any number of knobs, buttons, dials, or any suitable input device. The user inputs 517 may allow a patient or user to activate or initialize the sensor 500, select calibration information or request calibration, select or view the value of the physiological parameter monitored by the sensor 500, etc.

To facilitate communication with the sensor 502, the portable electronic device 500 may include a sensor interface 518. In one embodiment, the sensor interface may be a sensor board manufactured by Nellcor Puritan Bennett LLC. The sensor interface 518 may include various components configured to control, monitor, and send or receive signals from the sensor 502 and its components. For example, the sensor interface 518 may provide timing control signals and control when the emitter 504 is illuminated, and if multiple light sources are used, the multiplexed timing for the different light sources. The sensor interface 518 may also control the gating-in of signals from detector 506 through an amplifier 520. These signals may be sampled at the proper time, depending upon which of multiple light sources is illuminated, if multiple light sources are used.

The sensor 502 having an emitter 504 and a detector 506 may also include an encoder 524 that provides signals to allow the portable electronic device 500 to select appropriate calibration coefficients. As mentioned above, the encoder 524 may, for instance, be a coded resistor, EEPROM or other coding devices (such as a capacitor, inductor, PROM, RFID, a barcode, parallel resonant circuits, or a calorimetric indicator) that may provide a signal to the processor 510 related to the characteristics of the sensor 502 that may allow the processor 510 to determine the appropriate calibration characteristics for the sensor 502. Further, the encoder 524 may include encryption coding that prevents a disposable part of the sensor 500 from being recognized by a portable electronic device 500 or sensor interface 518 that is not able to decode the encryption. Such encryption coding is described in U.S. Pat. No. 6,708,049, which is hereby incorporated by reference in its entirety for all purposes.

Based on the received signals corresponding to the light received by detector 506, microprocessor 510 may calculate the value of the physiological parameter concentration using various algorithms. These algorithms may utilize coefficients, which may be empirically determined, corresponding to, for example, the wavelengths of light used. In some embodiments, these calibration coefficients may be stored in a ROM 526 of the portable electronic device 500. In a two-wavelength system, the particular set of coefficients chosen for any pair of wavelength spectra may be determined by the value indicated by the encoder 524 corresponding to a particular light source in a particular sensor 10. In one embodiment, multiple resistor values may be assigned to select different sets of coefficients. In another embodiment, the same resistors may be used to select from among the coefficients appropriate for an infrared source paired with either a near red source or far red source. Alternatively, in some embodiments, the sensor 502 may store calibration coefficients in the encoder 524.

Various embodiments of the portable electronic device 500 and sensor 502 may divide processing of the received signals from the patient 508 in different configurations. For example, in the embodiment illustrated in FIG. 6, the sensor interface 518 is a part of the portable electronic device 502. Accordingly, the portable electronic device 500 may receive unprocessed signals from the detector 506 and amplifier 520, and the sensor interface 518 may perform the requisite processing. In other embodiments, the sensor 502 may include a sensor interface to perform the processing of the signals received from the detector. In such an embodiment, the portable electronic device may receive processed signals corresponding to the actual value of the physiological parameter being measured. In this embodiment, no further processing of the signal may be performed by the portable electronic device 500. In yet other embodiments, the sensor interface may be disposed in a sensor cable connecting the sensor 502 to the portable electronic device 500.

What is claimed is:

1. A system, comprising:
a sensor capable of monitoring a physiological parameter, wherein the sensor is further capable of communicating with a portable electronic device capable of communicating over a wireless network.

2. The system of claim 1, wherein the sensor is operably coupled to the portable electronic device.

3. The system of claim 1, wherein the sensor is capable of communicating with the portable electronic device via a wireless communication protocol.

4. The system of claim 1, wherein the sensor is capable of communicating with the portable electronic device via a sensor cable.

5. The system of claim 1, wherein the sensor comprises a pulse oximetry sensor, an electrocardiogram sensor, a blood glucose sensor, blood pressure sensor, and/or a temperature sensor and/or any combination thereof.

6. The system of claim 1, wherein the portable electronic device comprises a cellular phone, a personal data assistant, a media player, a GPS device, and/or a wireless handheld device, and/or combinations thereof.

7. The sensor of claim 1, wherein the portable electronic device comprises a memory capable of storing calibration information for the sensor.

8. The sensor of claim 1, wherein the wireless network comprises a cellular network.

9. The sensor of claim 1, wherein the wireless network comprises a wide area network, and/or a local network.

10. A method of operating a sensor, comprising
sensing a physiological parameter of a patient; and
providing a signal based at least in part upon the physiological parameter to a portable electronic device capable of communicating over a wireless network.

11. The method of claim 10, further comprising providing calibration information to the portable electronic device.

12. The method of claim 10, further comprising transferring data to the portable electronic device via an advanced programming interface executed on the portable electronic device.

13. The method of claim 12, wherein the advanced programming interface comprises Binary Runtime Environment for Wireless.

14. The method of claim 12, wherein the advanced programming interface comprises Java Micro Edition.

15. The method of claim 11, comprising receiving calibration information from the portable electronic device.

16. A method comprising:
receiving data from a sensor capable of sensing a physiological parameter;
determining the physiological parameter from the data; and
communicating the physiological parameter to a portable electronic device capable of communicating over a wireless network.

17. The method of claim 16, comprising storing the physiological parameter in a memory.

18. The method of claim 16, comprising providing calibration information to the sensor.

19. The method of claim 16, comprising sending the physiological parameter to a remote location via the wireless communication network.

20. The method of claim 16, comprising sending the physiological parameter to a caregiver via the wireless communication network.
